(19) United States
(12) Patent Application Publication
(10) Pub. No.: US 2013/0144053 A1
(43) Pub. Date: Jun. 6, 2013

(54) Process for the Production of Seven-Membered Lactam Morphinans

(71) Applicant: Mallinckrodt LLC, Hazelwood, MO (US)

(72) Inventors: Christopher W. Grote, Webster Groves, MO (US); Joseph P. McClurg, Manchester, MO (US); Frank W. Moser, Arnold, MO (US)

(73) Assignee: Mallinckrodt LLC, Hazelwood, MO (US)

(21) Appl. No.: 13/705,205

(22) Filed: Dec. 5, 2012

Related U.S. Application Data
(60) Provisional application No. 61/566,763, filed on Dec. 5, 2011.

(51) Int. Cl. C07D 491/18

(52) U.S. Cl. CPC C07D 491/18
USPC 540/520

(57) ABSTRACT

The present invention relates to improved processes for preparing lactam morphinans. The processes generally transform keto-morphinans to seven-membered lactam morphinans using a hydroxyamine sulfonic acid reagent and proceed in high yield and with good selectivity.

### CROSS REFERENCE TO RELATED APPLICATIONS

This application claims the benefit of U.S. Provisional Application No. 61/566,763 filed Dec. 5, 2011, which is incorporated herein in its entirety.

### FIELD OF THE INVENTION

The present invention relates to improved processes for preparing lactam morphinans. The processes generally transform keto-morphinans to seven-membered lactam morphinans using a hydroxyamine sulfonic acid reagent.

### BACKGROUND OF THE INVENTION

Morphinan compounds are important pharmaceuticals showing a variety of activity. Modifications to the core morphinan structure may show increased or varying biological activities. Specifically, modification of the core ring structure is a desirable scaffold for new therapeutics. Several ring enlargement reactions are known for expanding small molecules with few functionalities, however, their application to morphinans has had limited success. For example, the Schmidt reaction is a ring expansion reaction that utilizes a hydrazoic acid. Rather than expansion of the ring structure in the morphinan compound, the Schmidt reaction results in cleavage of the morphinan ether ring. Beckmann rearrangements have also been attempted in morphinans. Generally, the Beckmann reaction proceeds from an oxime which is then contacted with an acid to give an amide or lactam. Previous attempts to utilize a Beckmann rearrangement on morphinan oximes resulted in poor yields and mixtures of products.

Thus, there remains a need for processes for the production of seven-membered lactams with high selectivity and in good yields.

### SUMMARY OF THE INVENTION

The present invention relates to a process for producing seven-membered lactam morphinans.

In one aspect, the present invention provides a process for producing a seven-membered lactam morphinan. The process comprises contacting a keto-morphinan with a hydroxyamine sulfonic acid to form the seven-membered lactam morphinan.

In one iteration, the keto-morphinan is a compound comprising Formula (I) and the seven-membered lactam morphinan is a compound comprising Formula (III):

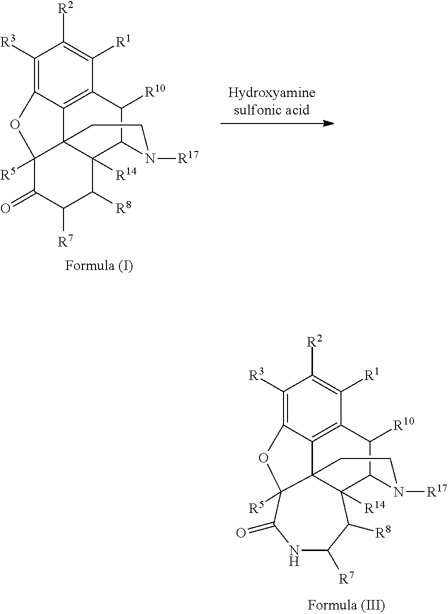

wherein:

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl; and

R¹⁷ is chosen from hydrogen, hydrocarbyl, and substituted hydrocarbyl.

In another iteration, the keto-morphinan is a compound comprising Formula (II) and the seven-membered lactam morphinan is a compound comprising Formula (IV):

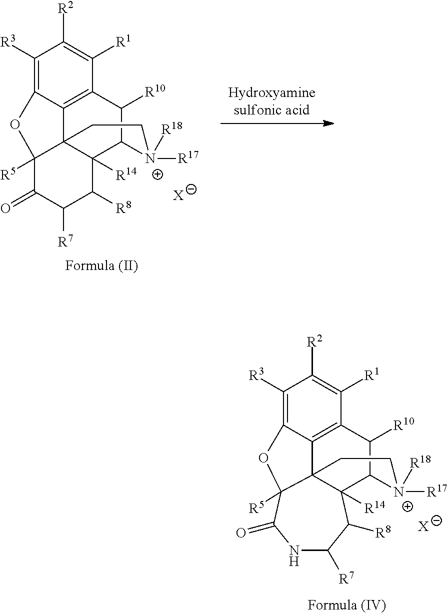

wherein

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl;

R¹⁷ is chosen from hydrogen, hydrocarbyl, and substituted hydrocarbyl;

R¹⁸ is chosen from hydrogen, hydrocarbyl, and substituted hydrocarbyl; and

X is chosen from fluorine, chlorine, bromine, and iodine.

Other features and iterations of the disclosure are described in more detail herein.

### DETAILED DESCRIPTION OF THE INVENTION

Briefly, therefore, the present invention relates to the synthesis of seven-membered lactam morphinans using a hydroxyamine sulfonic acid. The process produces seven-membered lactam morphinans in high yields and with few undesired byproducts. Moreover, the reaction proceeds from the keto-morphinan without the isolation of an oxime intermediate, resulting in a more facile transformation of the keto-morphinan to the seven-membered lactam.

The core morphinan structure generally consists of the fused ring structure shown below. The structure below shows the numbering associated with individual atoms of the alkaloid ring structure. The processes described herein result in a modification of the core structure. The process results in an expansion of a six-membered ring to a seven-membered ring. The core structure can be substituted as described herein. These compounds have stereocenters, and thus, each stereocenter may have an R or an S configuration such that both C-15 and C-16 are on the same side of the molecule.

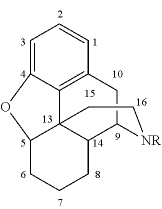

### (I). Reaction Conditions

Generally, the processes for producing the seven-membered lactam morphinans comprise contacting a keto-morphinan with a hydroxyamine sulfonic acid. In some embodiments, the process further comprises a work-up with a proton donor or an organic solvent such that the seven-membered lactam may be isolated from the reaction mixture.

Keto-morphinans are morphinan compounds having a ketone group. Keto-morphinans may be naturally occurring morphinans or may be synthetically prepared. In preferred embodiments, the keto-morphinan is a 6-keto-morphinan, meaning that the carbon atom of the ketone group is at the 6-position of the core morphinan structure. In some aspects of the invention, the keto-morphinan is a compound comprising Formula (I):

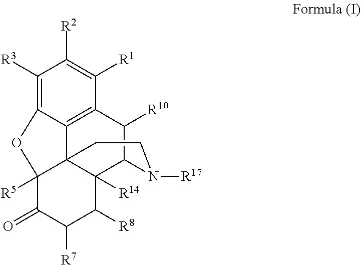

wherein

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl; and

R¹⁷ is chosen from hydrogen, hydrocarbyl, and substituted hydrocarbyl.

In some embodiments, R¹, R², R⁵, R⁷, R⁸, and R¹⁰ are hydrogen; R³ and R¹⁴ are selected from hydrogen, {—}OH and {—}OCH₃; and R¹⁷ is selected from allyl, cyclopropylmethyl, and methyl. In one embodiment, R¹, R², R⁵, R⁷, R⁸, R¹⁰ and R¹⁴ are hydrogen; R³ is {—}OCH₃; and R¹⁷ is methyl. In another embodiment, R¹, R², R⁵, R⁷, R⁸, and R¹⁰ are hydrogen; R³ is hydroxyl; R¹⁴ is hydroxyl; and R¹⁷ is cyclopropylmethyl. In still another embodiment, R¹, R², R⁵, R⁷, R⁸, and R¹⁰ are hydrogen; R³ is hydroxyl; R¹⁴ is hydroxyl; and R¹⁷ is methyl. In a further embodiment, R¹, R², R⁵, R⁷, R⁸, and R¹⁰ are hydrogen; R³ is hydroxyl; R¹⁴ is hydroxyl; and R¹⁷ is allyl.

In other aspects of the invention, the keto-morphinan is a compound comprising Formula (II):

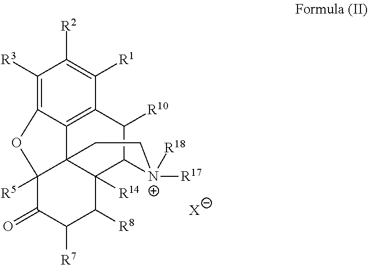

wherein:

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl;

R¹⁷ is chosen from hydrogen, hydrocarbyl, and substituted hydrocarbyl;

R¹⁸ is chosen from hydrogen, hydrocarbyl, and substituted hydrocarbyl; and

X is chosen from fluorine, chlorine, bromine, and iodine.

In some embodiments, R¹, R², R⁵, R⁷, R⁸, and R¹⁰ are hydrogen; R³ is hydroxyl; R¹⁴ is hydroxyl; R¹⁷ is cyclopropylmethyl; R¹⁸ is methyl; and X is bromine.

In some embodiments, the keto-morphinan may have a particular stereochemical configuration. Generally, keto-morphinans have at least four stereocenters at C-5, C-9, C-13, and C14. The C-5, C-9, C-13, and C-14 carbons of the keto-morphinans, may be either R or S, so long as both C-15 and C-16 are on the same side of the molecule. In some embodiments, the C-5, C-9, C-13, and C-14 stereocenters of the keto-morphinans are chosen from RRRR, RRRS, RRSR, RRSS, RSRS, RSRR, RSSR, RSSS, SRRR, SRRS, SRSR, SRSS, SSRS, SSRR, SSSR, and SSSS, respectively. In another aspect, the C-5, C-9, C-13, and C-14 stereocenters of the keto-morphinans are chosen from RRSR, SRSR, RSRS, and SSRS, respectively. In another embodiment, the C-5, C-9, C-13, and C-14 stereocenters of the keto-morphinans are RRSR, respectively. In still another embodiment, the C-5, C-9, C-13, and C-14 stereocenters of the keto-morphinans are SSRS, respectively. In some aspects of the invention, the keto-morphinans are (+)-morphinans. In other aspects of the invention, the keto-morphinans are (−)-morphinans. In exemplary embodiments, the keto-morphinan is selected from (−)-hydrocodone, (+)-hydrocodone, (−)-naloxone, (+)-naloxone, (−)-naltrexone, (+)-naltrexone, (−)-naltrexone methyl bromide, (+)-naltrexone methyl bromide, (−)-oxycodone, and (+)-oxycodone.

The process further comprises contacting the keto-morphinan with a hydroxyamine sulfonic acid. A hydroxyamine sulfonic acid comprises both a hydroxyamine group and a sulfonic acid group including various salts thereof. Salts may be any known in the art, including, but not limited to sodium, potassium, and lithium salts. In one embodiment, the hydroxyamine sulfonic acid comprises the compound HON(SO₃Na)₂. In a preferred embodiment, the hydroxyamine sulfonic acid is hydroxyamine-O-sulfonic acid, H₂NOSO₂OH.

In some embodiments, the keto-morphinan and the hydroxyamine sulfonic acid are combined in a mole-to-mole ratio ranging from about 1:0.5 to about 1:10, respectively. In an alternate embodiment, the keto-morphinan and the hydroxyamine sulfonic acid are combined in a mole-to-mole ratio of about 1:1 to about 1:5, respectively. In other embodiments, the keto-morphinan and the hydroxyamine sulfonic acid are combined in a mole-to-mole ratio ranging from about 1:1 to about 1:2, from about 1:2 to about 1:3, from about 1:3 to about 1:4, or from about 1:4 to about 1:5, respectively. In an exemplary embodiment, the keto-morphinan and the hydroxyamine sulfonic acid are combined in a mole-to-mole ratio of about 1:2, respectively. In another exemplary embodiment, the keto-morphinan and the hydroxyamine sulfonic acid are combined in a mole-to-mole ratio of about 1:1.5, respectively

The reaction mixture may further comprise one or more solvents. The solvent can and will vary depending on the substrates used in the process. The solvent may be a protic solvent, an aprotic solvent, a non-polar solvent, or combinations thereof. Suitable examples of protic solvents include, but are not limited to, methanol, ethanol, isopropanol, n-propanol, isobutanol, n-butanol, s-butanol, t-butanol, formic acid, acetic acid, water and combinations thereof. Non-limiting examples of suitable aprotic solvents include acetonitrile, diethoxymethane, N,N-dimethylacetamide (DMAC), N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl)ether, 1,4-dioxane, N-methyl-2-pyrrolidinone (NMP), ethyl formate, formamide, hexamethylphosphoramide, N-methylacetamide, N-methylformamide, methylene chloride, nitrobenzene, nitromethane, propionitrile, sulfolane, tetramethylurea, tetrahydrofuran (THF), 2-methyl tetrahydrofuran, trichloromethane, and combinations thereof. Suitable examples of non-polar solvents include, but are not limited to, alkane and substituted alkane solvents (including cycloalkanes), aromatic hydrocarbons, esters, ethers, combinations thereof, and the like. Specific non-polar solvents that may be employed include, for example, benzene, butyl acetate, t-butyl methylether, chlorobenzene, chloroform, chloromethane, cyclohexane, dichloromethane, dichloroethane, diethyl ether, ethyl acetate, diethylene glycol, fluorobenzene, heptane, hexane, isopropyl acetate, methyltetrahydrofuran, pentyl acetate, n-propyl acetate, tetrahydrofuran, toluene, and combinations thereof. When one or more organic solvents are present in the reaction the solvents may be present in any ratio without limitation. In one preferred embodiment, for example, the solvent may be a 96% solution of formic acid in water.

In general, the weight ratio of the solvent to the keto-morphinan may range from about 0.5:1 to about 100:1. In various embodiments, the weight ratio of the solvent to the keto-morphinan may range from 0.5:1 to about 5:1, from about 5:1 to about 25:1, or from about 25:1 to about 100:1. In preferred embodiments, the weight ratio of the solvent to the keto-morphinan may range from about 2:1 to about 10:1.

In some embodiments, the reaction may comprise an additional proton donor. The proton donor generally has a pKa less than about 6. Suitable proton donors having this characteristic include, but are not limited to, acetic acid, formic acid, methane sulfonic acid, phosphoric acid, sulfuric acid, hydrochloric acid, hydrobromic acid, hydroiodic acid, trifluoromethane sulfonic acid, toluenesulfonic acid, and the like.

The molar ratio of the keto-morphinan to the proton donor may range from about 1:0.5 to about 1:100. In various embodiments, the molar ratio of the keto-morphinan to the proton donor may range from 1:10 to about 1:80, or from about 1:20 to about 1:60. In some embodiments, the molar ratio of the keto-morphinan to the proton donor may range be about 1:1, or about 1:5, or about 1:10, or about 1:20, or about 1:30, or about 1:40, or about 1:50, or about 1:60, or about 1:80, or about 1:100. In an exemplary embodiment, the molar ratio of the keto-morphinan to the proton donor may be about 1:40.

The reaction between the keto-morphinan and the hydroxyamine sulfonic acid may be conducted at a variety of temperatures ranging from about −5° C. to about 100° C. depending on the substrate and the temperature may vary over the course of the reaction. For instance, the reaction may be conducted at a about 20° C., or about 25° C., or about 30° C., or about 35° C., or about 40° C., or about 45° C., or about 50° C., or about 55° C., or about 60° C., or about 65° C., or about 70° C., or about 75° C., or about 80° C., or about 85° C., or about 90° C., or about 95° C., or about 100° C., or about 105° C., or about 110° C., or about 115° C. In various embodiments, the reaction may be conducted at a temperature ranging from about 20° C. to about 30° C. In one exemplary embodiment, the reaction may be conducted at a temperature of about 25° C.

Generally, the reaction between the keto-morphinan and the hydroxyamine sulfonic acid is allowed to proceed for a sufficient period of time until the reaction is substantially complete. Reaction completeness may be determined by any method known to one skilled in the art, such as chromatography (e.g., TLC, HPLC, or LC). The duration of the reaction may range from about 2 hours to more than 5 days. In some embodiments, the reaction may be allowed to proceed for about 6 hours, about 12 hours, about 18 hours, about 24 hours, about 36 hours, about 48 hours, about 60 hours, about 72 hours, or about 84 hours. In this context, a “completed reaction” generally means that the reaction mixture contains a significantly diminished amount of the keto-morphinan. Typically, the amount of the keto-morphinan remaining in the reaction mixture may be less than about 10%, or more preferably less than about 5%.

In some aspects of the invention, the reaction between the keto-morphinan and the hydroxyamine sulfonic acid reagent results in a sulfonated imine intermediate. A sulfonated imine as used herein refers to an imine group N-substituted with a sulfonic acid group. In some aspects, where the hydroxyamine sulfonic acid is hydroxyamine-O-sulfonic acid and the keto-morphinan is a 6-keto-morphinan, the intermediate comprises the compound of Formula (I)(a), below:

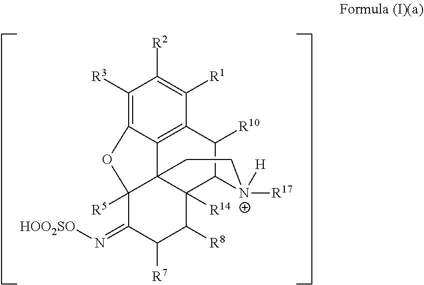

wherein:

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl; and

R¹⁷ is chosen from hydrogen, hydrocarbyl, and substituted hydrocarbyl.

In an alternative embodiments, where the hydroxyamine sulfonic acid is hydroxyamine-O-sulfonic acid and the keto-morphinan is a 6-keto-morphinan, the intermediate comprises the compound of Formula (II)(a), below:

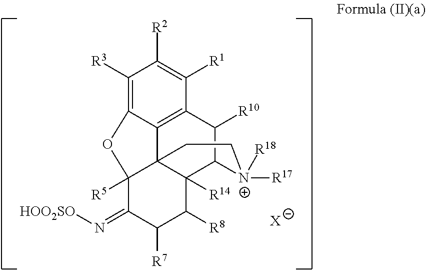

wherein:

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl;

R¹⁷ is chosen from hydrogen, hydrocarbyl, substituted hydrocarbyl;

R¹⁸ is chosen from hydrogen, hydrocarbyl, and substituted hydrocarbyl; and

X is chosen from fluorine, chlorine, bromine, and iodine.

### (II). Work-Up

The processes may further comprise one or more work-up steps to obtain the seven-membered lactam morphinan. In some embodiments, the intermediate compound may be converted to the seven-membered lactam morphinan by addition of a proton acceptor. In general, the proton acceptor will have a pKa greater than about 9. Suitable proton acceptors having this characteristic include ammonia, borate salts (such as, for example, NaBO₃), bicarbonate salts (such as, for example, NaHCO₃, KHCO₃, LiCO₃, and the like), carbonate salts (such as, for example, Na₂CO₃, K₂CO₃, Li₂CO₃, and the like), hydroxide salts (such as, for example, NaOH, KOH, and the like), organic bases (such as, for example, pyridine, methylamine, diethylamine, triethylamine, diisopropylethylamine, N-methylmorpholine, N,N-dimethylaminopyridine), and mixtures of any of the above. In preferred embodiments, the proton acceptor may be ammonia, ammonium hydroxide, potassium hydroxide, or sodium hydroxide. In an exemplary embodiment, the proton acceptor may be ammonia.

The proton acceptor may be added in a solvent. The solvent can be added before, after, or at the same time as the proton donor. In some embodiments, the proton acceptor may be present in an aqueous solution. In such embodiments, the concentration of the proton acceptor in water may vary from about a VA v/v solution to about a 99% v/v solution. In other embodiments, the concentration of the proton acceptor in water may vary from about a 20% v/v solution to about a 60% v/v solution. In other aspects, the concentration of the proton acceptor in water may vary from about a 20% v/v solution to about a 30% v/v solution. In one embodiment, the concentration of the proton acceptor in water is about a 29% v/v solution. In an exemplary embodiment, the proton acceptor may be an aqueous solution of about 29% of ammonia in water.

The total amount of proton acceptor added to work up the reaction can and will vary. In some embodiments, a proton acceptor is added until the pH of the reaction mixture is above 9. In other embodiments, the proton acceptor is added until the pH of the reaction mixture is about 9, or about 9.2, or about 9.4, or about 9.6.

In another embodiment, the reaction is worked-up through addition of an organic solvent. The organic solvent may be added to the reaction in any amount, In some embodiments, the organic solvent is added in excess to the reaction mixture. In general, the weight ratio of the keto-morphinan to the organic solvent may range from about 1:10 to about 1:100. In various embodiments, the weight ratio of the keto-morphinan to the organic solvent may range from 1:1 to about 1:5, from about 1:5 about 1:25, or from about 1:25 to about 1:100. In preferred embodiments, the weight ratio of the keto-morphian to the organic solvent is about 1:50. The organic solvent may be selected from those listed in Section (I). In an exemplary embodiment, the organic solvent is acetone.

In various aspects, work up of the reaction occurs at temperatures ranging from about −10° C. to about 50° C. In some aspects, the formation of the seven-membered lactam occurs at about −5° C., or at about 0° C., or at about 5° C., or at about 10° C., or at about 20° C., or at about 30° C. In various embodiments, formation of the seven-membered lactam occurs over 1 hour to about 1 day.

Generally, work-up of the reaction gives a precipitant which may be filtered, washed, and dried as is known in the art. The seven-membered lactam morphinan may be used as the crude precipitant or may be further purified by techniques including through extraction, chromoatogoraphy, filtration, evaporation, crystallization and drying (including vacuum, oven, and through chemical reagents) and the like.

The yield of the seven-membered lactam morphinan can and will vary. Typically, the yield of the seven-membered lactam morphinan will be at least about 60%. In one embodiment, the yield of the seven-membered lactam morphinan may range between about 60% and about 80%. In another embodiment, the yield of the seven-membered lactam morphinan may range between about 80% and about 90%. In a further embodiment, the yield of the seven-membered lactam morphinan may range between about 90% and about 95%. In still another embodiment, the yield of the seven-membered lactam morphinan may be greater than about 95%.

The seven-membered lactam morphinan may be used or it may be converted to another compound using techniques familiar to those of skill in the art. For example, the seven-membered lactam morphinan may be converted into a pharmaceutically acceptable salt or may be further chemically derivatized.

The seven-membered lactams may be produced with a high level of regioselectivity. When a reaction has the potential to result in more than one structural isomer, preferential production of a single isomer is called regioselectivity. Formation of a lactam from a keto-morphinan as described herein may result in nitrogen insertion at different positions. For example, formation of a lactam from a 6-keto-morphinan may result in nitrogen atom insertion between the 5 position and the carbonyl, or between the 7-position and the carbonyl as shown in Example 1. In some aspects of the invention, the reaction proceeds with a high level of regioselectivity. In some embodiments, the reaction produces a single regioisomer in a yield above about 70%, above about 75%, above about 80%, above about 85%, or above about 90%. In yet another embodiment, the reaction produces a single regioisomer in a yield above about 95%.

In one aspect, the process produces a compound comprising Formula (III) as shown in Reaction Scheme 1.

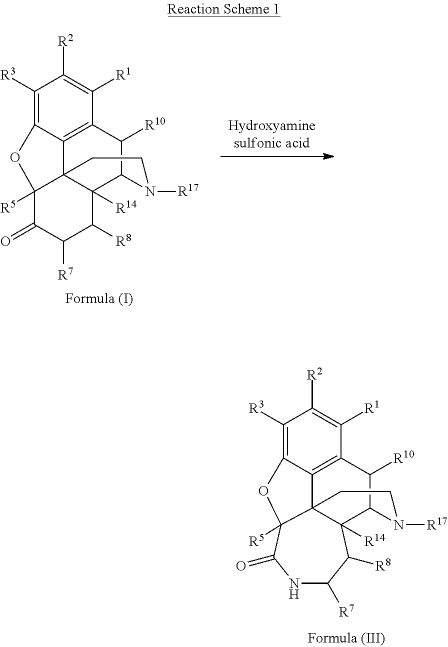

wherein

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl; and

R¹⁷ is chosen from hydrogen, hydrocarbyl, and substituted hydrocarbyl.

In some embodiments, R¹, R², R⁵, R⁷, R⁸, and R¹⁰ are hydrogen; R³ and R¹⁴ are selected from hydrogen, {—}OH and {—}OCH₃; and R¹⁷ is selected from allyl, cyclopropylmethyl, and methyl. In one embodiment, R¹, R², R⁵, R⁷, R⁸, R¹⁰ and R¹⁴ are hydrogen; R³ is {—}OCH₃; and R¹⁷ is methyl. In another embodiment, R¹, R², R⁵, R⁷, R⁸, and R¹⁰ are hydrogen; R³ is hydroxyl; R¹⁴ is hydroxyl; and R¹⁷ is cyclopropylmethyl. In still another embodiment, R¹, R², R⁵, R⁷, R⁸, and R¹⁰ are hydrogen; R³ is hydroxyl; R¹⁴ is hydroxyl; and R¹⁷ is methyl. In a further embodiment, R¹, R², R⁵, R⁷, R⁸, and R¹⁰ are hydrogen; R³ is hydroxyl; R¹⁴ is hydroxyl; and R¹⁷ is allyl.

In another aspect, the process produces a compound comprising Formula (IV) according to Reaction Scheme 2.

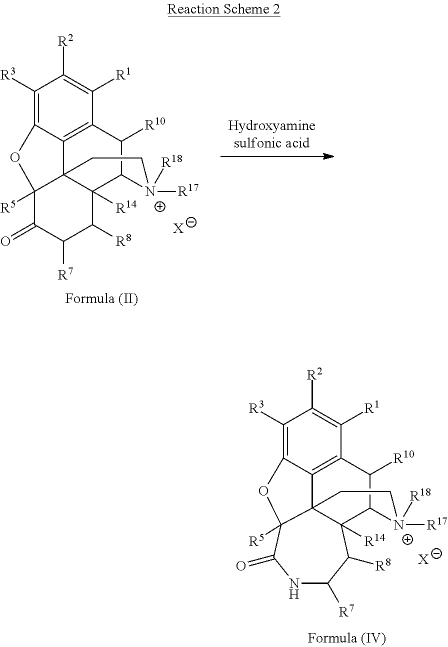

wherein

R¹, R², R³, R⁵, R⁷, R⁸, and R¹⁰ are independently chosen from hydrogen, hydrocarbyl, substituted hydrocarbyl, halogen, and {—}OR¹⁵;

R¹⁴ is chosen from hydrogen and {—}OR¹⁵;

R¹⁵ is chosen from hydrogen, hydrocarbyl, and substituted hydrocarbyl;

R¹⁷ is chosen from hydrogen, hydrocarbyl, and substituted hydrocarbyl;

R¹⁸ is chosen from hydrogen, hydrocarbyl, and substituted hydrocarbyl; and

X is chosen from fluorine, chlorine, bromine, and iodine.

In some embodiments, R¹, R², R⁵, R⁷, R⁸, and R¹⁰ are hydrogen; R³ is hydroxyl; R¹⁴ is hydroxyl; R¹⁷ is cyclopropylmethyl; R¹⁸ is methyl; and X is bromine.

In another aspect, the reaction may occur with stereoselectivity. In one embodiment, the reaction comprises an amount of a single enantiomer greater than about 50%, or greater than about 60%, or greater than about 70%, or greater than about 80%, or greater than about 90%.

The seven-membered lactam morphinans may have a (−) or a (+) orientation with respect to the rotation of polarized light. More specifically, each chiral center of the morphinan may have an R or an S configuration. In some embodiments, the seven-membered lactam morphinans have at least four chiral centers C-5, C-9, C-13, and C-14. Thus, the configurations C-5, C-9, C-13, and C-14, respectively, may be stereocenters of the lactam morphinans are chosen from RRRR, RRRS, RRSR, RRSS, RSRS, RSRR, RSSR, RSSS, SRRR, SRRS, SRSR, SRSS, SSRS, SSRR, SSSR, and SSSS, respectively. In another aspect, the C-5, C-9, C-13, and C-14 stereocenters of the seven-membered lactam-morphinans are chosen from RRSR, SRSR, RSRS, and SSRS, respectively. In another embodiment, the C-5, C-9, C-13, and C-14 stereocenters of the seven-membered lactam morphinans are RRSR, respectively. In still another embodiment, the C-5, C-9, C-13, and C-14 stereocenters of the seven-membered lactam morphinans are SSRS, respectively. In some aspects of the invention, the keto-morphinans are (+)-morphinans. In other aspects of the invention, the keto-morphinans are (−)-morphinans.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

The compounds described herein have asymmetric centers. Compounds of the present invention containing an asymmetrically substituted atom may be isolated in optically active or racemic form. All chiral, diastereomeric, racemic forms and all geometric isomeric forms of a structure are intended, unless the specific stereochemistry or isomeric form is specifically indicated.

The term “acyl,” as used herein alone or as part of another group, denotes the moiety formed by removal of the hydroxyl group from the group COON of an organic carboxylic acid, e.g., RC(O)—, wherein R is R¹, R¹O—, R¹R²N—, or R¹S—, R¹ is hydrocarbyl, heterosubstituted hydrocarbyl, or heterocyclo, and R² is hydrogen, hydrocarbyl, or substituted hydrocarbyl.

The term “acyloxy,” as used herein alone or as part of another group, denotes an acyl group as described above bonded through an oxygen linkage (O), e.g., RC(O)O— wherein R is as defined in connection with the term “acyl.”

The term “allyl,” as used herein not only refers to compound containing the simple allyl group (CH₂═CH—CH₂—), but also to compounds that contain substituted allyl groups or allyl groups forming part of a ring system.

The term “alkyl” as used herein describes groups which are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “alkenyl” as used herein describes groups which are preferably lower alkenyl containing from two to eight carbon atoms in the principal chain and up to 20 carbon atoms. They may be straight or branched chain or cyclic and include ethenyl, propenyl, isopropenyl, butenyl, isobutenyl, hexenyl, and the like.

The term “alkoxide” or “alkoxy” as used herein is the conjugate base of an alcohol. The alcohol may be straight chain, branched, cyclic, and includes aryloxy compounds.

The term “alkynyl” as used herein describes groups which are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic” as used herein alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar” as used herein alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The term “enrichment” means an amount above the statistical distribution if all chiral centers had an equal probability of being alpha or beta.

The terms “carbocyclo” or “carbocyclic” as used herein alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The terms “epoxy” or “epoxide” as used herein means a cyclic ether. The ring structure generally comprises from 2 to 5 carbon atoms in the ring.

The terms “halogen” or “halo” as used herein alone or as part of another group refer to chlorine, bromine, fluorine, and iodine.

The term “heteroatom” refers to atoms other than carbon and hydrogen.

The term “heteroaromatic” as used herein alone or as part of another group denotes optionally substituted aromatic groups having at least one heteroatom in at least one ring, and preferably 5 or 6 atoms in each ring. The heteroaromatic group preferably has 1 or 2 oxygen atoms and/or 1 to 4 nitrogen atoms in the ring, and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The terms “heterocyclo” or “heterocyclic” as used herein alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group preferably has 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phospho, nitro, and thio.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “protecting group” as used herein denotes a group capable of protecting a particular moiety, wherein the protecting group may be removed, subsequent to the reaction for which the protection is employed, without disturbing the remainder of the molecule. A variety of protecting groups and the synthesis thereof may be found in “Protective Groups in Organic Synthesis” by T. W. Greene and P. G. M. Wuts, John Wiley & Sons, 1999.

The “substituted hydrocarbyl” moieties described herein are hydrocarbyl moieties which are substituted with at least one atom other than carbon, including moieties in which a carbon chain atom is substituted with a heteroatom such as nitrogen, oxygen, silicon, phosphorous, boron, or a halogen atom, and moieties in which the carbon chain comprises additional substituents. These substituents include alkyl, alkoxy, acyl, acyloxy, alkenyl, alkenoxy, aryl, aryloxy, amino, amino, acetal, carbamyl, carbocyclo, cyano, ester, ether, halogen, heterocyclo, hydroxyl, keto, ketal, phospho, nitro, and thio.

A sulfonated imine as described herein is an imine group with a sulfur comprising group attached.

Having described the invention in detail, it will be apparent that modifications and variations are possible without departing from the scope of the invention defined in the appended claims.

### EXAMPLES

### Example 1

### Preparation of a 7-Membered Lactam Morphinan with Thionyl Chloride from the Lactam Morphinan

The oxime derivative of naltrexone was added to a solution of 1,4-dioxane and thionyl chloride under room temperature. The resulting product was identified by LC/MS as a mixture of regioisomers as shown below.

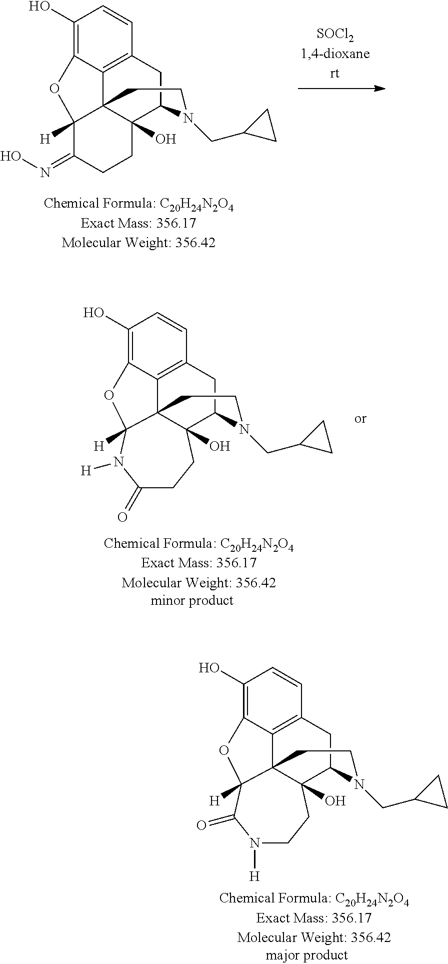 [SMILES: O/N=C1/CC[C@@]2(O)[C@H]3Cc4ccc(O)c5c4[C@@]2(CCN3CC2CC2)[C@H]1O5 ; O=C1CC[C@@]2(O)[C@H]3Cc4ccc(O)c5c4[C@@]2(CCN3CC2CC2)[C@H](N1)O5 ; O=C1NCC[C@@]2(O)[C@H]3Cc4ccc(O)c5c4[C@@]2(CCN3CC2CC2)[C@H]1O5]

### Example 2

### Preparation of a 7-Membered Lactam Morphinan with Tosyl Chloride

The oxime derivative of naltrexone was added to a solution of tosyl chloride in acetone. Sodium bicarbonate was added and the mixture was reacted at room temperature. LC/MS identified the following mixture of products.

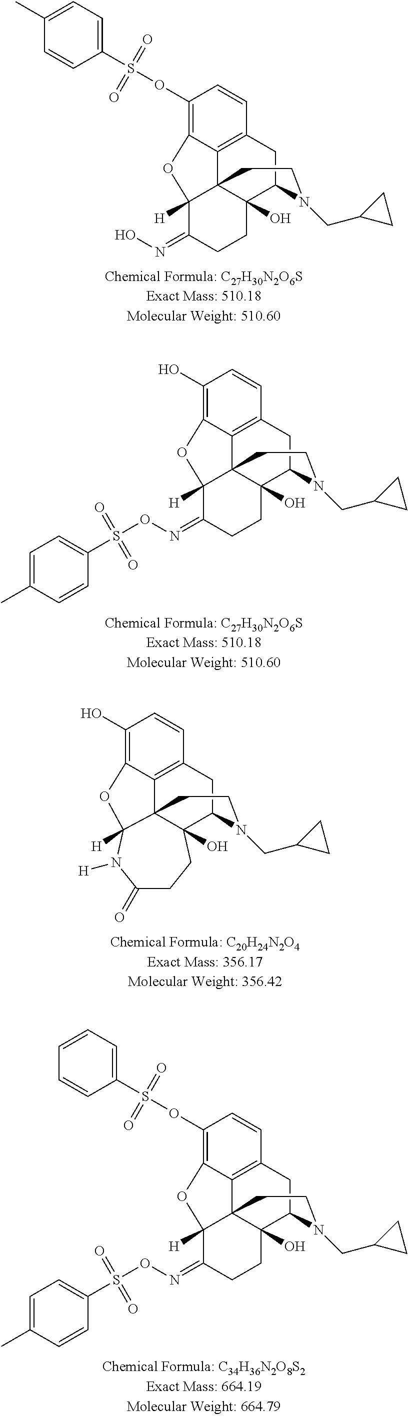 [SMILES: Cc1ccc(S(=O)(=O)Oc2ccc3c4c2O[C@H]2/C(=N\O)CC[C@@]5(O)[C@@H](C3)N(CC3CC3)CC[C@]425)cc1 ; Cc1ccc(S(=O)(=O)O/N=C2/CC[C@@]3(O)[C@H]4Cc5ccc(O)c6c5[C@@]3(CCN4CC3CC3)[C@H]2O6)cc1 ; O=C1CC[C@@]2(O)[C@H]3Cc4ccc(O)c5c4[C@@]2(CCN3CC2CC2)[C@H](N1)O5 ; Cc1ccc(S(=O)(=O)O/N=C2/CC[C@@]3(O)[C@H]4Cc5ccc(OS(=O)(=O)c6ccccc6)c6c5[C@@]3(CCN4CC3CC3)[C@H]2O6)cc1]

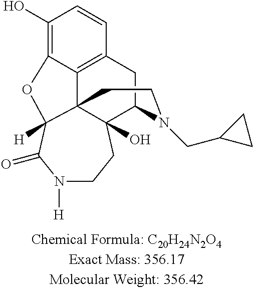 [SMILES: O=C1NCC[C@@]2(O)[C@H]3Cc4ccc(O)c5c4[C@@]2(CCN3CC2CC2)[C@H]1O5]

### Example 3

### Preparation of a 7-Memebered Lactam from (−)-Naltrexone

(−)-Naltrexone (2.36 g, 6.91 mmol) was dissolved in 98% formic acid (10 mL) at room temperature. The reaction was stirred for 15 minutes to ensure complete dissolution. Hydroxyamine O-sulfonic acid (1.95g, 17.3 mmol, 2.5 eq) was added all at once. The reaction stirred for 24 h at room temperature where the reaction was deemed complete by LC. The reaction mixture was added dropwise in 29% NH₃/H₂O at 5° C. This mixture was stirred at room temperature for 24 h. The precipitate was filtered off washing the precipitate with distilled water (25 mL). After sitting overnight at room temperature, additional product formed. The second precipitate was filtered, washed with distilled water (5.0 mL), and then dried on the funnel for 2 h. Combing both precipitates, drying the solids at 50° C. for 48 h under vacuum yielded the product (2.02g, 5.7 mmol, 82% yield).

### Example 4

### Preparation of a 7-Memebered Lactam from (−)-Hydrocodone

(−)-Hydrocodone (2.15 g, 7.18 mmol) was dissolved in 98% formic acid (10 mL) at room temperature. Hydroxyamine O-sulfonic acid (1.22 g, 10.8 mmol, 1.5 eq) was added all at once. The reaction stirred for 24 h at room temperature where the reaction was deemed complete by LC. To the solution was added distilled water (50 mL) then the solution was cooled between 0° C. and 5° C. The pH was adjusted to 9.4 using 29% NH3/H₂O added dropwise. A precipitate formed. After cooling for 1 h at 0° C. to 5° C., the precipitate was filtered, washed with distilled water (20 mL), and dried on the funnel for 1 h. The solid was transferred to a drying dish and dried at 40° C. for 48 h under vacuum yielded the product (2.05 g, 6.5 mmol, 91% yield).

### Example 5

### Preparation of a 7-Memebered Lactam from (−)-Oxycodone

(−)-Oxycodone (2.46 g, 7.8 mmol) was dissolved in 96% formic acid (10 mL) at room temperature. This mixture was stirred for 15 minutes to ensure complete dissolution. Hydroxyamine O-sulfonic acid (2.21 g, 19.5 mmol, 2.5 eq) was added all at once. The reaction stirred for 72 h at room temperature where the reaction was deemed complete by LC. To the solution was added distilled water (50 mL) then the solution was cooled between 0° C. and 5° C. The pH was adjusted to 9.4 using 29%NH₃/H₂O added dropwise. A precipitate formed. After cooling for 1 h at 0° C. to 5° C., the precipitate was filtered, washed with distilled water (10 mL), and dried on the funnel for 1 h. The solid was transferred to a drying dish and dried at 45° C. for 24 h under vacuum yielded the product (1.91 g, 5.8 mmol, 74% yield).

### Example 6

### Preparation of a 7-Memebered Lactam from (−)-Naltrexone Methyl Bromide

(−)-Naltrexone Methyl Bromide (1.06 g, 2.43 mmol) was dissolved in 96% formic acid (5.0 mL) at room temperature. Hydroxyamine O-sulfonic acid (0.69 g, 6.07 mmol, 2.5 eq) was added all at once. The reaction stirred for 5 days at room temperature where the reaction was deemed complete by LC. To the solution was added acetone (5.0 mL) then the solution was cooled between 0° C. and 5° C. No precipitate occurred. The solvent was removed under reduced pressure on the rotovap. Acetone (50 mL) was added and the mixture was stirred overnight at room temperature. A precipitate formed. The precipitate was filtered, washed with acetone (25 mL), and dried on the funnel for 1 h. The solid was transferred to a drying dish and dried at 50° C. for 48 h under vacuum yielded the product (0.86 g, 1.8 mmol, 76% yield).

### Example 7

### l Preparation of a 7-Memebered Lactam from (−)-Naloxone

(−)-Naloxone (2.86 g, 8.7 mmol) was dissolved in 96% formic acid (10 mL) at room temperature. This mixture was stirred for 15 minutes to ensure complete dissolution. Hydroxyamine O-sulfonic acid (1.48 g, 13.1 mmol, 1.5 eq) was added all at once. The reaction stirred for 24 h at room temperature where the reaction was deemed complete by LC. The reaction mixture was added dropwise into 29% NH₃/H₂O (6.0 mL) maintained at 5° C. A precipitate formed. After stirring for 4 h at 0° C. to 5° C., the precipitate was filtered, washed with distilled water (20 mL), and dried on the funnel. The solid was transferred to a drying dish and dried at 45° C. for 24 h under vacuum yielded the product (2.45 g, 7.2 mmol, 82% yield).

### Example 8

### Preparation of a 7-Memebered Lactam from (+)-Naloxone

(+)-Naloxone (0.46 g, 1.41 mmol) was dissolved in 96% formic acid (4.0 mL) at room temperature. This mixture was stirred for 15 minutes to ensure complete dissolution. Hydroxyamine O-sulfonic acid (0.278 g, 2.46 mmol, 1.75 eq) was added all at once. The reaction stirred for 24 h at room temperature where the reaction was deemed complete by LC. The reaction mixture was added dropwise into a cold solution of 29% NH₃/H₂O. A precipitate formed and stirred for 3 h at 5° C. The precipitate was isolated by filtration, washed with distilled water (2×25 mL), and then dried on the funnel. The filtrate was extracted with CHCl₃ (3×20 mL). The extracts were combined, dried over anhydrous MgSO₄ (˜1.0 g), filtered, and evaporated. The product was isolated by gravity SiO₂ chromatography (G60, 70-230 mesh) eluting with a gradient from 50% EtOAc/heptane to 100% EtOAc. Combination of the desired fractions, evaporation, then drying in a vacuum oven at 40 C for 48 h yielded the product (400 mg, 1.16 mmol, 83% yield) as a foam.

### Example 9

### Preparation of a 7-Memebered Lactam from (+)-Oxycodone

(+)-Oxycodone (1.88 g, 7.8 mmol) was dissolved in 96% formic acid (10 mL) at room temperature. This mixture was stirred for 15 minutes to ensure complete dissolution. Hydroxyamine O-sulfonic acid (1.34 g, 11.9 mmol, 2.0 eq) was added all at once. The reaction stirred for 24 h at room temperature where the reaction was deemed complete by LC. To the solution was added distilled water (50 mL) then the solution was cooled to 25° C. The pH was adjusted to 9.2 using 29% NH₃/H₂O added dropwise. A gummy precipitate formed. The solution was extracted using CHCl3 (2×50 mL). Extracts were combined, dried over anhydrous MgSO₄ (˜2.0 g), filtered, and evaporated. The product was isolated by gravity SiO₂ chromatography (G60, 70-230 mesh) eluting with a gradient from 0% MeOH/CHCl₃ to 5% MeOH/CHCl₃. Combination of the desired fractions, evaporation, then drying in a vacuum oven at 40° C. for 48 h yielded the product (1.67 mg, 5.1 mmol, 85% yield) as an off-white foam.

### Example 10

### Preparation of a 7-Memebered Lactam from (+)-Naltrexone

(+)-Naltrexone (1.53 g, 4.48 mmol) was dissolved in 96% formic acid (10 mL) at room temperature. This mixture was stirred for 15 minutes to ensure complete dissolution. Hydroxyamine O-sulfonic acid (1.01 g, 8.96 mmol, 2.0 eq) was added all at once. The reaction stirred for 48 h at room temperature where the reaction was deemed complete by LC. To the solution was added distilled water (50 mL) then the solution was cooled to 25° C. The pH was adjusted to 9.2 using 29% NH₃/H₂O added dropwise. A gummy precipitate formed. The solution was extracted using CHCl₃ (3×50 mL). Extracts were combined, dried over anhydrous MgSO₄ (˜2.0 g), filtered, and evaporated. The product was isolated by gravity SiO₂ chromatography (G60, 70-230 mesh) eluting with a gradient from 0% MeOH/CHCl₃ to 3% MeOH/CHCl₃. Combination of the desired fractions, evaporation, then drying in a vacuum oven at 40° C. for 48 h yielded the product (1.42 g, 4.0 mmol, 89% yield) as an off-white foam.

What is claimed is:

1. A process for the production of a seven-membered lactam morphinan, wherein the process comprises contacting a keto-morphinan with a hydroxyamine sulfonic acid to form the seven-membered lactam morphinan.

2. The process of claim 1, wherein the hydroxyamine sulfonic acid is hydroxyamine-O-sulfonic acid; and the keto-morphinan and the hydroxyamine sulfonic acid are present in a mole-to-mole ratio from about 1:1 to about 1:5.

3. The process of claim 1, wherein the contacting is performed in the presence of a proton donor; and the keto-morphinan and the proton donor are present in a mole-to-mole ratio from about 1:10 to about 1:80.

4. The process of claim 1, wherein the process is conducted at a temperature ranging from about 0° C. to about 50° C.

5. The process of claim 1, wherein the process further comprises addition of a proton acceptor; the proton acceptor is present in an aqueous solution; and
the aqueous solution comprises from about 20% to 60% v/v of the proton acceptor.

6. The process of claim 1, wherein a single regioisomer of the seven-membered lactam morphinan has a yield above about 75%.

7. The process of claim 1, wherein the seven-membered lactam morphinan is a (+)-morphinan or a (−)-morphinan.

8. The process of claim 1, wherein the keto-morphinan is a 6-keto-morphinan comprising Formula (I) and the seven-membered lactam morphinan comprises Formula (Ill): 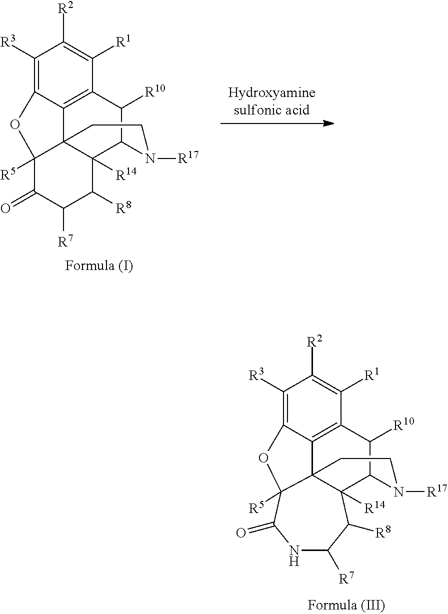
wherein:
R¹, R², R³, R⁵, R⁷, R⁸, and R¹⁰ are independently chosen from hydrogen, hydrocarbyl, substituted hydrocarbyl, halogen, and {—}OR¹⁵;
R¹⁴ is chosen from hydrogen and {—}OR¹⁵;
R¹⁵ is chosen from hydrogen, hydrocarbyl, and substituted hydrocarbyl; and
R¹⁷ is chosen from hydrogen, hydrocarbyl, and substituted hydrocarbyl.

9. The process of claim 8, wherein R¹, R², R⁵, R⁷, R⁸, R¹⁰ and R¹⁴ are hydrogen; R³ is {—}OCH₃; and R¹⁷ is methyl.

10. The process of claim 8, wherein R¹, R², R⁵, R⁷, R⁸, and R¹⁰ are hydrogen; R³ is hydroxyl; R¹⁴ is hydroxyl; and R¹⁷ is methyl, cyclopropylmethyl, or allyl.

11. The process of claim 8, wherein the hydroxyamine sulfonic acid is hydroxyamine-O-sulfonic acid and an intermediate compound comprising Formula (I)(a) is formed: 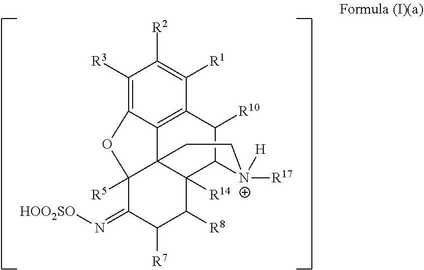
wherein
R¹, R², R³, R⁵, R⁷, R⁸, and R¹⁰ are independently chosen from hydrogen, hydrocarbyl, substituted hydrocarbyl, halogen, and {—}OR¹⁵;
R¹⁴ is chosen from hydrogen and {—}OR¹⁵;
R¹⁵ is chosen from hydrogen, hydrocarbyl, and substituted hydrocarbyl; and
R¹⁷ is chosen from hydrogen, hydrocarbyl, substituted hydrocarbyl.

12. The process of claim 11, wherein the compound comprising Formula (I) and the hydroxyamine sulfonic acid are present in a mole-to-mole ratio from about 1:1 to about 1:5; the contacting is performed in the presence of a proton donor; the mole-to-mole ratio of the compound comprising Formula (I) to the proton donor is from about 1:10 to about 1:80; the process is conducted at a temperature ranging from about 0° C. to about 50° C.; the process further comprises addition of a proton acceptor; the proton acceptor is present in an aqueous solution; and the aqueous solution comprises from about 20% to about 60% v/v of the proton acceptor.

13. The process of claim 12, wherein ratio of the compound comprising Formula (I) to the hydroxyamine sulfonic acid is about 1:1.5; the proton donor is formic acid; the ratio of the compound comprising Formula (I) to the proton donor is about 1:40; the reaction is conducted at about 25° C.; the proton acceptor is an aqueous solution of about 29% v/v of ammonia in water.

14. The process of claim 12, wherein the compound comprising Formula (III) is a (+)-morphinan or a (−)-morphinan; and C-5, C-9, C-13, and C-14 of the compound comprising Formula (III) have a configuration chosen from RRRR, RRRS, RRSR, RRSS, RSRS, RSRR, RSSR, RSSS, SRRR, SRRS, SRSR, SRSS, SSRS, SSRR, SSSR, and SSSS, respectively, provided that both C-15 and C-16 are on the same side of the molecule.

15. The process of claim 1, wherein the keto-morphinan is a 6-keto-morphinan comprising Formula (II) and the seven-membered lactam morphinan comprises Formula (IV): 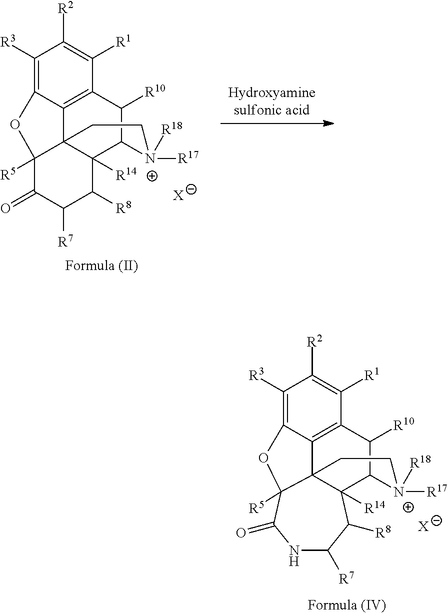
wherein:
R¹, R², R³, R⁵, R⁷, R⁸, and R¹⁰ are independently chosen from hydrogen, hydrocarbyl, substituted hydrocarbyl, halogen, and {—}OR¹⁵;
R¹⁴ is chosen from hydrogen and {—}OR¹⁵;
R¹⁵ is chosen from hydrogen, hydrocarbyl, and substituted hydrocarbyl;
R¹⁷ is chosen from hydrogen, hydrocarbyl, and substituted hydrocarbyl;
R¹⁸ is chosen from hydrogen, hydrocarbyl, and substituted hydrocarbyl; and
X is chosen from fluorine, chlorine, bromine, and iodine.

16. The process of claim 15, wherein R¹, R², R⁵, R⁷, R⁸, and R¹⁰ are hydrogen; R³ is hydroxyl; R¹⁴ is hydroxyl; R¹⁷ is cyclopropylmethyl; R¹⁸ is methyl; and X is bromine.

17. The process of claim 15, wherein the hydroxyamine sulfonic acid is hydroxyamine-O-sulfonic acid and an intermediate compound comprising compound comprising Formula (II)(a) is formed: 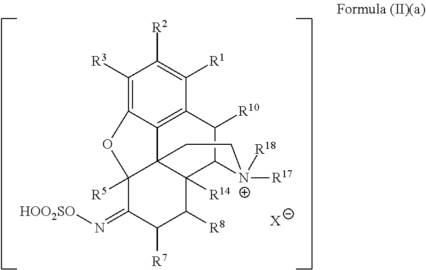
wherein
R¹, R², R³, R⁵, R⁷, R⁸, and R¹⁰ are independently chosen from hydrogen, hydrocarbyl, substituted hydrocarbyl, halogen, and {—}OR¹⁵;
R¹⁴ is chosen from hydrogen and {—}OR¹⁵;
R¹⁵ is chosen from hydrogen, hydrocarbyl, and substituted hydrocarbyl;
R¹⁷ is chosen from hydrogen, hydrocarbyl, substituted hydrocarbyl;
R¹⁸ is chosen from hydrogen, hydrocarbyl, and substituted hydrocarbyl; and
X is chosen from fluorine, chlorine, bromine, and iodine.

18. The process of claim 17, wherein the compound comprising Formula (II) and the hydroxyamine sulfonic acid are present in a mole-to-mole ratio from about 1:1 to about 1:5; the contacting is performed in the presence of a proton donor; the compound comprising Formula (II) and the proton donor are present in a mole-to-mole ratio from about 1:10 to about 1:80; the process is conducted at a temperature ranging from about 0° C. to about 50° C.; the process further comprises addition of a proton acceptor; the proton acceptor is present in an aqueous solution; and the aqueous solution comprises from about 20% to about 60% v/v of the proton acceptor.

19. The process of claim 18, wherein ratio of the compound comprising Formula (II) to the hydroxyamine sulfonic acid is about 1:1.5; the proton donor is formic acid; the ratio of the compound comprising Formula (II) to the proton donor is about 1:40; the reaction is conducted at about 25° C.; the proton acceptor is an aqueous solution of about 29% v/v of ammonia in water.

20. The process of claim 18, wherein the compound comprising Formula (IV) is a (+)-morphinan or a (−)-morphinan; and C-5, C-9, C-13, and C-14 of the compound comprising Formula (IV) have a configuration chosen from RRRR, RRRS, RRSR, RRSS, RSRS, RSRR, RSSR, RSSS, SRRR, SRRS, SRSR, SRSS, SSRS, SSRR, SSSR, and SSSS, respectively, provided that both C-15 and C-16 are on the same side of the molecule.
